(19) United States
(12) Patent Application Publication
(10) Pub. No.: US 2007/0118187 A1
(43) Pub. Date: May 24, 2007

(54) ALERTING METHOD FOR A TRANSVASCULAR TISSUE STIMULATION SYSTEM

(76) Inventors: Stephen Denker, Mequon, WI (US); Arthur J. Beutler, Greendale, WI (US); Cherik Bulkes, Sussex, WI (US)

Correspondence Address: QUARLES & BRADY LLP, 411 E. WISCONSIN AVENUE, SUITE 2040, MILWAUKEE, WI, 53202-4497, US

(21) Appl. No.: 11/556,979

(22) Filed: Nov. 6, 2006

Related U.S. Application Data
(60) Provisional application No. 60/738,439, filed on Nov. 21, 2005.

(51) Int. Cl. A61N 1/00

(52) U.S. Cl. 607/60

(57) ABSTRACT

A system for stimulating tissue of the patient includes an implantable medical device and an external power source. The medical device receives and extracts electrical energy from a first wireless signal and has a detector circuit that senses a physiological characteristic of the patient. The sensing can occur simultaneously while electrical stimulation pulses are applied to the tissue. A feedback transmitter that sends information related to the physiological characteristic via a second wireless signal. The external power source transmits the first wireless signal and extracts the information from a second wireless signal. When the information indicates existence of a predefined condition, a communication module, that preferably includes a cellular telephone sends a message for reception by the remote monitor to alert medical personnel.

### CROSS-REFERENCE TO RELATED APPLICATIONS

This application claims benefit of U.S. Provisional Patent Application No. 60/738,439 filed Nov. 21, 2005.

### STATEMENT REGARDING FEDERALLY SPONSORED RESEARCH OR DEVELOPMENT

Not Applicable

### BACKGROUND OF THE INVENTION

1. Field of Invention

The present invention relates to implantable medical devices that electrically stimulate tissue for therapeutic purposes, and more particularly to communication of data regarding operation of the implanted device to external monitoring equipment.

2. Description of the Related Art

Various physiological ailments have remedies that involve implanting a stimulation device which applies electrical pulses to an organ or other part of the patient's body associated with the ailment. The stimulation device includes an electronic pulse generator from which electrical leads extend to electrodes in contact with bodily tissue, which when electrically stimulated provide therapy to the patient.

For example, a common remedy for people with slowed or disrupted natural heart activity is to implant a cardiac pacing device, which is a small electronic apparatus that stimulates the heart to beat at regular rates. The pacing device typically is implanted in the patient's chest and has sensor electrodes that detect electrical impulses associated with heart contractions. These sensed impulses are analyzed to determine when abnormal cardiac activity occurs, in which event a pulse generator is triggered to produce electrical pulses. Wires carry these pulses to electrodes placed adjacent specific cardiac muscles, that when electrically stimulated contract the heart chambers.

U.S. Pat. No. 7,003,350 describes a cardiac pacemaker that is implanted in the vasculature of the patient. A power transmitter, located outside the patient, emits a radio frequency signal that is received by a pacing circuit on a stent embedded in a vein or artery near the patient's heart. The radio frequency signal induces a voltage pulse in an antenna of the pacing circuit, thereby conveying electrical power to the implanted circuitry. The pacing circuit senses electrical activity of the heart and determines when to apply that electrical power in the form of voltage pulses across a pair of electrodes in contact with blood vessel walls. The voltage pulses stimulate adjacent muscles, thereby contracting the heart.

These stimulation devices need to monitor and/or confirm overall treatment performance and efficacy. A cardiac pacing device, for example, monitors whether the pacing pulses are effective in improving or correcting heart rhythm. Other physiological parameters can be sensed to gather statistical data continuously or periodically which data can be compared against a baseline.

It is desired that physiological and device performance data be communicated from the implanted device to equipment outside the patient for review by medical personnel. It is further desirable that medical personnel be alerted automatically when the communicated data indicates adverse conditions. For example, the user and medical personnel must be alerted if the power transmitter is inadvertently removed or improperly positioned, so that the implanted device does not receive the radio frequency signal that provides operating power to the device.

### SUMMARY OF THE INVENTION

The present system monitors an implanted medical device that stimulates tissue of a patient. This system can be configured to perform one or more alerting functions which include: warning the patient or a caregiver to perform action to correct an adverse condition detected by the monitoring, provide verification of proper placement of the medical device, and autonomously initiate communication with external, remotely located equipment.

The system for monitoring a medical patient and stimulating the patient's tissue includes a medical device for implantation entirely in vasculature of the patient and an external power source that is outside the patient. The medical device has a discriminator that receives and extracts energy from a first wireless signal which is used to power the medical device. A detector circuit produces data regarding a physiological characteristic or performance of the medical device and a feedback transmitter that sends information related to the data via a second wireless signal. That information can comprise the data or information derived from processing and analysis of the data.

The external power source transmits the first wireless signal and has a receiver that receives and extracts the information from a second wireless signal. A communication module is provided for communicating with a remote monitor. When the information indicates existence of a predefined condition, the communication module sends an alert message via a third wireless signal for reception by the remote monitor.

In one embodiment, the communication module has cellular telephone circuitry that produces the third wireless signal. When the data indicates existence of the predefined condition, the communication module dials a telephone number assigned to a remote monitor and sends an alert message for reception by the remote monitor.

In another aspect of the present invention, the medical device has a pair of electrodes for contacting the patient's tissue and a stimulation circuit applies electrical stimulation pulses to the pair of electrodes. The detector circuit also is connected to the pair of electrodes and senses a physiological characteristic of the medical patient simultaneously when an electrical stimulation pulse is being applied to those electrodes. In a preferred embodiment of this aspect, the detector circuit has an instrumentation amplifier with a variable gain and inputs connected to the pair of electrodes. The instrumentation amplifier is dynamically configured to have a lower gain while a stimulation pulse is being applied to the pair of electrodes than at other times.

### BRIEF DESCRIPTION OF DRAWINGS

FIG. 1 is an illustration of a tissue stimulation system attached to a medical patient;

FIG. 2 is an isometric, cut-away view of a patient's blood vessels in which a receiver antenna, a stimulator, and an electrode of an intravascular medical device have been implanted at different locations; and

FIG. 3 is a schematic circuit diagram of the external and internal components for the tissue stimulation system.

### DETAILED DESCRIPTION OF THE INVENTION

Although the present invention is being described in the context of and implanted tissue stimulation system and specifically a cardiac pacing system, it can be used with other implanted medical devices. Furthermore, the inventive concepts are not limited to devices implanted in the vascular system, but can be employed with components implanted elsewhere in the animal.

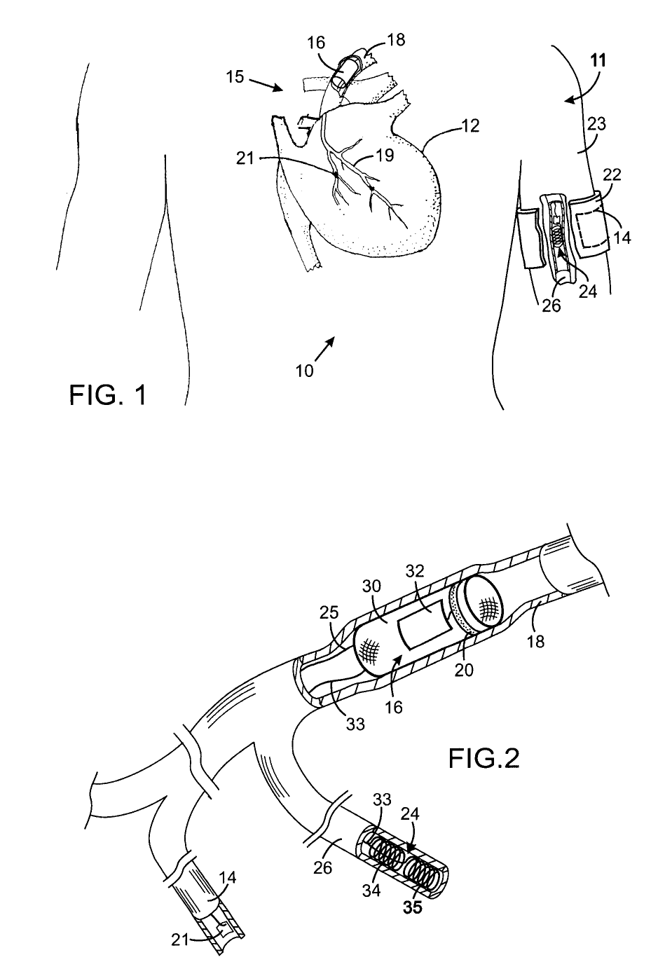

Initially referring to FIG. 1, a tissue stimulation system 10 for electrically stimulating a heart 12 to contract comprises an external power source 14 and a medical device 15 implanted in the blood circulatory system of a human medical patient 11. The medical device 15 receives a radio frequency (RF) signal from the power source 14 worn outside the patient and the circuitry of the implanted device is electrically powered from the energy of that signal. At appropriate times, the medical device 15 delivers an electrical stimulation pulse into the surrounding tissue of the patient.

The power source 14 includes a radio frequency transmitter that is powered by a battery. The transmitter periodically emits a signal at a predefined radio frequency that is applied to a transmitter antenna in the form of a coil of wire within a band 22 that is placed around the patient's upper arm 23. The radio frequency is received by an antenna assembly 24 implanted in the basilic vein 26 of the patient's upper right arm 23, for example. In a basic version of the tissue stimulation system 10, the radio frequency signal merely conveys energy for powering the medical device 15 implanted in the patient. In other systems, the transmitter modulates the radio frequency signal with commands that configure or control the operation of the medical device 15.

Referring to FIGS. 1 and 2, the exemplary implanted medical device 15 includes an intravascular stimulator 16 located a vein or artery 18 in close proximity to the heart. Because of its electrical circuitry, the stimulator 16 is relatively large requiring a blood vessel that is larger than the arm vein 26, that is approximately five millimeters in diameter. Therefore, the stimulator 16 is implanted in the superior or inferior vena cava, for example. However, it is contemplated that further miniaturization of components will reduce the size of the stimulator circuitry enabling placement in smaller veins and arteries. Electrical wires lead from the stimulator 16 through the cardiac vascular system to one or more locations in smaller blood vessels 19, such as the coronary sinus vein, at which stimulation of the heart is desired. At those locations, the electrical wire 25, from the stimulation circuit 32 is connected to a remote electrode 21 secured to the blood vessel wall.

Because the stimulator 16 of the medical device 15 is near the heart and relatively deep in the chest of the medical patient 11, the RF antenna assembly 24 is implanted in a vein or artery 26 of the patient's upper right arm 23 at a location surrounded by the transmitter antenna within the arm band 22. That arm vein or artery 26 is significantly closer to the skin and thus implanted antenna assembly 24 picks up a greater amount of the energy from the first radio frequency signal emitted by the power source 14, than if that antenna assembly was located on the stimulator 16. Alternatively, another limb, the neck or other area of the body with an adequately sized blood vessel close to the skin surface of the patient can be used. The implanted antenna assembly 24 comprises a receiver antenna 34 and a transmitter antenna 35 in the form of wire coils that are connected to the stimulator 16 by a cable 33.

As illustrated in FIG. 2, the intravascular stimulator 16 has a body 30 constructed similar to well-known expandable vascular stents commonly employed to enlarge constricted blood vessels. The stimulator body 30 comprises a plurality of interwoven wires formed to have a memory defining a tubular shape or envelope. Those wires are heat-treated platinum, Nitinol, a Nitinol alloy wire, stainless steel, plastic wires or other materials which will provide the shape memory and not react with the tissue at the implantation site. Plastic or substantially nonmetallic wires may be loaded with a radiopaque substance to be visible with conventional fluoroscopy. The stimulator body 30 has a memory so that it normally assumes an expanded configuration when unconfined, but is capable of assuming a collapsed configuration when disposed and confined within a catheter assembly. In that collapsed state, the tubular body 30 has a relatively small diameter enabling it to pass freely through the vasculature of a patient while being guided on the catheter assembly. After being positioned in the desired blood vessel, the body 30 is released from the catheter assembly and expands to engage the blood vessel wall. The stimulator body 30 and other components of the medical device 15 are implanted in the patient's vasculature.

The body 30 has a stimulation circuit 32 mounted thereon and if implanted proximate the heart 12, holds a first electrode 20 in the form of a ring that encircles the body. Alternatively, when the stimulator 16 is distant from the heart 12, the first electrode 20 is remotely located in a small cardiac blood vessel, much the same as a second electrode 21. Conventional circuitry within the stimulation circuit 32 detects the electrical activity of the heart 12 and determines when electrical pulses need to be applied so that the heart contracts at the proper rate. When stimulation is desired, the stimulation circuit 32 applies electrical voltage from an internal storage device across the electrodes 20 and 21. The second electrode 21 and the first electrode, when located remotely from the stimulator 16, can be mounted on a collapsible body of the same type as the stimulator body 30. In all the examples cited with regard to the FIG. 2, it should be understood that the exemplary size limit is driving the decision on the placement of components. It is contemplated that miniaturization of components will lead to many more options for component placement.

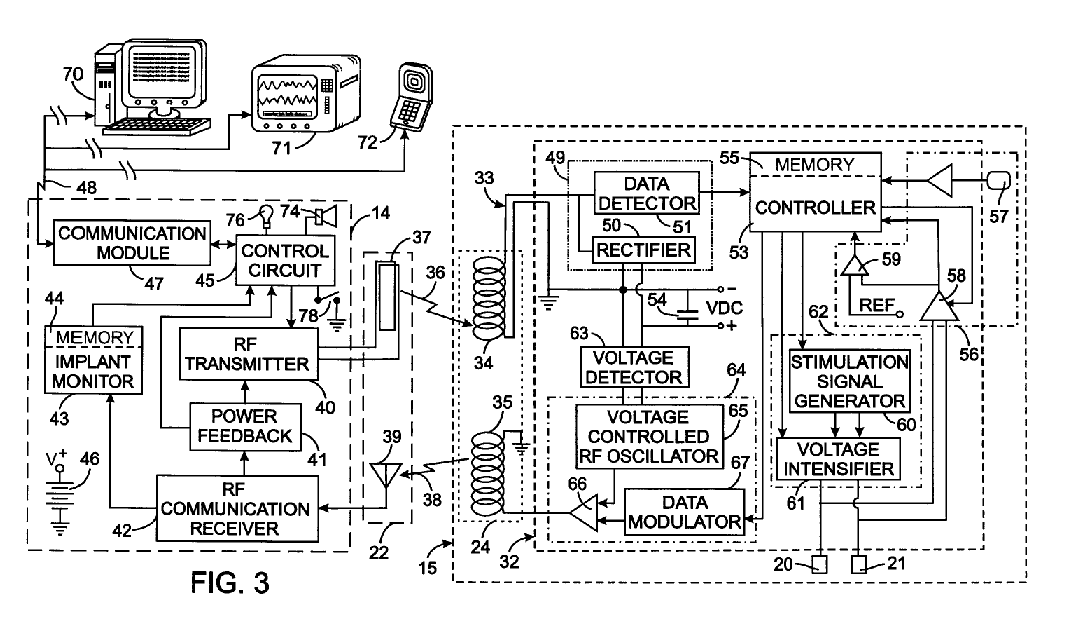

Referring to FIG. 3, the electrical circuitry for the external power source 14 of the tissue stimulation system 10 includes a battery 46, a radio frequency (RF) power transmitter 40, a power feedback module 41, an RF communication receiver 42, an implant monitor 43, and a control circuit 45. In addition, a communication module 47 is provided to exchange data and commands via a communication link 48 with a remote monitor, such as a personal computer 70, patient monitor 71, cellular telephone 72, pager, personal digital assistant (PDA), or similar wireless equipment. The communication link 48 preferably is wireless, such as a radio frequency signal or a cellular telephone call.

The battery 46 is rechargeable allowing patient mobility with periodic recharge cycles. Depending upon the type and size of the battery, the time between recharge cycles may be days, months or years. Power transmitter 40 and a first antenna 37 periodically transmit a radio frequency first wireless signal 36 that is pulse width modulated (PWM) in a variably controlled manner to convey different amounts of energy to the implanted medical device 15. The stimulation circuit 32 is connected to the receiver antenna 34 that is tuned to pick-up the first wireless signal 36 which also carries control commands to the medical device 15. The receiver antenna 34 is coupled to a discriminator 49 that separates the received signal into electrical power and commands. A rectifier 50 in the discriminator 49 extracts energy from the first wireless signal. Specifically, the radio frequency, first wireless signal 36 is rectified to produce a DC voltage (VDC) that is applied across the storage device 54, e.g. a capacitor, which functions as a power supply by furnishing electrical power to the other components of the medical device.

The charge of the power storage device 54 is monitored and the stimulation circuit 32 sends data indicating its power needs via a second wireless signal 38 at a different radio frequency. The second wireless signal is received by a second antenna 39 and the RF communication receiver 42 in the external power source 14. A power feedback module 41, connected to the communication receiver, is part of closed control loop that receives the medical device's power needs data and responds by controlling the duty cycle of the first wireless signal 36 to ensure that the medical device 15 has a sufficient amount of electrical power.

As necessary, the first wireless signal 36 also carries control commands that specify operational parameters of the medical device 15, such as the duration of the stimulation pulses to be applied to the electrodes 20 and 21. These commands are sent digitally as a series of binary bits encoded on the first wireless signal 36 by fixed duration pulses of that signal. The receiver antenna 34 also is coupled to a data detector 51 within discriminator 49 that recovers the commands and other data from the first wireless signal. The recovered information is sent to a controller 53, which controls the operation of a stimulation circuit 62. Preferably, the controller 53 comprises a microcomputer that has analog and digital input/output circuits and an internal memory 55 that stores a software control program and data acquired and used by that program.

The controller 53 also receives signals from a detector circuit 56, which includes a sensor 57 and an amplifier, that detect physiological characteristics, such as temperature, blood pressure, blood flow, blood volume, and blood glucose level of the patient 11. The physiological data is stored by the controller 53 in the memory 55 from which it is periodically read and communicated to the external power source 14 or another external data gathering device.

The first and second electrodes 20 and 21 detect electrical activity of the heart and provide conventional electrocardiogram signals that are applied to inputs of a variable gain instrumentation amplifier 58 that also is part of the detector circuit 56. The gain of the instrumentation amplifier 58 is varied by a signal from the controller 53, as will be described. The output of the instrumentation amplifier 58 is coupled to an analog input of the controller 53 and to an input of a differentiator 59. The differentiator 59 has another input which receives a reference level (REF) which enables signal transition detection to provide a signal to the controller 53 indicating events in the sensed cardiac activity. For example, the differentiator 59 in conjunction with software executed by the controller 53 determines the heart rate based on the number of transitions counted over a defined time interval. The controller 53 commences cardiac pacing when the heart rate goes out of a normal range for a given length of time. When the heart rate indicates fibrillation, the controller initiates defibrillation pulse to the electrodes 20 and 21. A histogram of the electrocardiogram signals and pacing data related to usage of the medical device is stored in memory 55.

Stimulation Signal Regulation

The software executed by the controller 53 analyzes the electrocardiogram signals from the first and second electrodes 20 and 21 and the other physiological signals from the sensors 57 to determine when and how to stimulate the patient's heart. When stimulation is required the controller 53 issues a command designating the voltage level, shape, and duty cycle of stimulation pulses to be applied to the first and second electrodes 20 and 21. That command is sent to a stimulation signal generator 60 which responds by applying one or more pulses of voltage from the storage device 54 across the electrodes. The stimulation signal generator 60 controls the intensity and shape of the pulses. The output pulses from the stimulation signal generator 60 can be applied either directly to the first and second electrodes 20 and 21 or via an optional voltage intensifier 61. The voltage intensifier 61 preferably is a “flying capacitor” inverter that charges and discharges in a manner that essentially doubles the power. However, other kinds of devices can be used to increase the stimulation voltage.

The first and second electrodes 20 and 21 also are used as sensors to provide feedback signals for regulating the stimulation. When stimulation is occurring, the instrumentation amplifier 58 has low gain (1× or lower) to avoid saturation and thus sense a physiological data simultaneously while a stimulation pulse is occurring. This is particularly useful to determine the impedance of the tissue between the electrodes 20 and 21. The low gain setting allows measurement of the tissue and electrode interface impedance by using the known stimulation pulse duration and amplitude as a known source and the system impedance as a known impedance. From the sensed voltage and the known impedances, the tissue and electrode interface impedance can be determined. This information can also be logged into the memory 55 over time to monitor physiological changes that may occur.

When stimulation is inactive, the instrumentation amplifier 58 has a normal gain (100×-200×) to sense physiological characteristics, such as the electrical activity of the heart. At these times, the controller 53 analyzes the sensed physiological characteristics to calculate the actual heart rate and determine whether the heart is beating at the desired rate in response to pacing stimulation. If the heart is at the desired rate, the controller 53 decreases the stimulation pulse energy in steps until stimulation is no longer effective. The stimulation pulse energy then is increased until the desired rate occurs. Energy reduction is accomplished at least in two ways: (1) preferably, the duty cycle is reduced to linearly decrease that amount of energy dissipated in the tissue, or (2) the voltage amplitude is reduced in situations where energy dissipation might vary non-linearly because the tissue/electrode interface is unknown.

The stimulation is controlled by a functionally closed feedback loop. When stimulation commences, the sensed signal waveform can show a physiological response confirming effectiveness of that stimulation pulse. By stepwise increasing the stimulation pulse duration (duty cycle), a threshold can be reached in successive steps. When the threshold is reached, an additional duration can be added to provide a level of insurance that all pacing will occur above the threshold, or it may be sufficient to hold the stimulation pulse duration at the threshold.

After each successful stimulation pulse, a determination is made regarding the difference in duration existing between the last non-effective pulse and the present effective pulse. That difference in duration is added to the present time. The system then senses the effectiveness of subsequent stimulation pulses and remains at the same level for either an unlimited duration or backs off one step in pulse duration. When the effectiveness is maintained again after a preset time window, which could be a number of beats, minutes or hours, the system backs off one decrement at a time. As soon as the effectiveness of the stimulation pulses is lost, the system keeps incrementing the duration until an effective pulse is obtained. In summary, the sensing and stimulation is a closed loop system with two feedback responses: the first response is following an effective pulse and involves gradual reduction of duration after a predetermined number of beats or a predetermined time interval; and the second response is to an ineffective pulse and is immediate with pulse duration adjustment occurring within one beat.

Supplied Power Control

Another feedback control loop is employed to regulate the electrical power supplied to the implanted medical device 15 from the external power source 14. As mentioned previously, the rectifier 50 in the discriminator 49 of the medical device 15 extracts energy from the received radio frequency first wireless signal 36 to charge the storage device 54. The storage device 54 preferably is a super capacitor that is an electrochemical double layer capacitor (EDLC) hybrid between a conventional capacitor and a battery, and has a greater extend the life span and power capability than standard rechargeable batteries. However, a rechargeable battery can be employed as the storage device 54 instead of a capacitor. In either case, the circuitry of the medical device 15 receives power for an extended period even if the power source 14 is removed from the patient for short periods.

The DC voltage produced by rectifier 50 is regulated. For this function, the DC voltage is applied to a voltage detector 63 that senses and compares the DC voltage to a nominal voltage level desired for powering the medical device 15. The result of that comparison is a control voltage which indicates the relationship of the actual DC voltage derived from the first wireless signal 36 to the nominal voltage level. The control voltage is fed to a feedback transmitter 64 and specifically to the input of a voltage controlled radio frequency oscillator 65 which produces an output signal at a radio frequency that varies as a function of the control voltage. For example, the radio frequency oscillator 65 has a center, or second frequency from which the actual output frequency varies in proportion to the polarity and magnitude of the control signal and thus deviation of the actual DC voltage from the nominal voltage level. For example, the radio frequency oscillator 65 has a first frequency of 100 MHz and varies 100 kHz per volt of the control voltage deviation with the polarity of the control voltage determining whether the oscillator frequency decreases or increases from the second frequency. For this exemplary oscillator, if the nominal voltage level is five volts and the output of the rectifier 50 is four volts, or one volt less than nominal, the output of the voltage controlled, radio frequency oscillator 65 is 99.900 MHz (100 MHz-100 kHz). That output is applied by an RF amplifier 66 to the transmitter antenna 35 in the implanted antenna assembly 24 which emits the second RF wireless signal 38.

To control the energy of the first wireless signal 36, the power source 14 contains a second antenna 39 that picks up the second wireless signal 38 from the implanted medical device 15. Because the second wireless signal 38 indicates the level of energy received by medical device 15, this enables power source 14 to determine whether medical device requires more or less energy to be adequately powered. The second wireless signal 38 is sent from the second antenna 39 to the power feedback module 41 which detects the frequency shift of that wireless signal from the second frequency and thus the thus deviation of the actual DC voltage from the nominal voltage level, which is an ERROR signal. That ERROR signal is used to control the duty cycle of the pulses of the first wireless signal 36 and thus the amount of energy that signal provides to the medical device 15. By maintaining a constant voltage across storage device 54 in the medical device 15, it is ensured that only the needed amount of power is transmitted.

Physiological Sensing

Referring still to FIG. 3, the first and second electrodes 20 and 21 detect electrocardiogram signals representing electrical activity of the heart and the sensors 57 provide signals related to other physiological characteristics, such as temperature, blood pressure, blood flow, blood volume, and blood glucose level. More sophisticated data analysis also can be performed to detect cardiac abnormalities, such as arrhythmias and atrial fibrillation. The controller 53 of the implanted medical device 15 receives and digitizes those signals and stores the resultant data in memory 55. The sensors 57 may produce a signal that directly indicates a physiological characteristic, such as temperature or pressure, or the sensor signal may be processed in the controller 53 by software that implements a conventional algorithm to derive data, such as blood volume or blood glucose level, from that signal. Other data pertaining to operational conditions of the stimulation circuit 32 also are stored.

The data may be stored as trending logs that indicate patient and/or device conditions over time. Trending logs can be accumulated continuously with the implant monitor 43 keeping the highest time resolution for the most recent events in minutes, mid-range events in hours, and long-range events in days, weeks, etc. For example, it may be desired to take blood pressure readings every few minutes, whereas blood glucose levels can be recorded once an hour. In some instances the raw sensor data is averaged during a predefined time period by the controller and only the average is stored in the memory 55. For other kinds of data, only a maximum or minimum value occurring in a given time period is retained. The storage time resolution for a given kind of data also may vary depending upon the recency of each item of that data, wherein more recently acquired items have a higher resolution than older items in order to conserve storage space in the memory. For example, every blood pressure reading acquired at five minute intervals during the last hour are held in the memory, and the data more than an hour old is culled with only every sixth data item (one per half hour) being retained. Alternatively, the culling process may average groups of data items (e.g. six blood pressure readings) and keep only the average in memory. The storage procedures, such as storage time resolution, averaging, etc., are user configurable by commands entered into the personal computer 70 and transmitted by the power source 14 via the radio frequency first wireless signal 36 to the implanted medical device 15.

Alternatively, minimal data retention can occur in the implanted medical device 15 with the power source 14 performing the primary storage of data. Here the data acquired by the implanted medical device 15 is streamed in real-time via the radio frequency second wireless signal 38 to the power source 14 where the data is stored in the memory 44 of the implant monitor 43 or a memory of the control circuit. The raw sensor data can be sent for analysis by the implant monitor 43 to derive more complex data, such as blood volume and blood glucose level, and to detect cardiac abnormalities, such as arrhythmias and atrial fibrillation. Trend analysis also is performed on the raw sensor data and the complex data.

Regardless of the data processing and storage capacity of the implanted medical device 15, data at some point in time is communicated to the power source 14 or another data gathering device that is external to the patient 11. That data transfer may be at regular intervals based on a timer implemented by the controller 53, upon the data having a predefined characteristic, e.g. blood pressure above a defined level or atrial fibrillation occurring, or in response to a request sent by the power source 14. The request sent from the power source 14 may originate in its control circuit 45 or be relayed from the personal computer 70 or other remote monitor. When such transfer is initiated, the data is retrieved from the memory 55 in the medical device 15 and sent to a data modulator 67. The data modulator 67 formats the data into a message packet that is applied to the RF amplifier 66, which amplitude modulates the radio frequency signal from the voltage controlled RF oscillator 65 with that data packet. The modulated radio frequency signal is applied to the implanted transmitter antenna 35 from which it is emitted as the second wireless signal 38.

When the power source 14 receives the second wireless signal 38, the RF communication receiver 42 extracts modulated data which is transferred to the implant monitor 43 for storage in memory 44 and possible further processing. The power source 14 may also forward the data to the remote monitor, e.g. personal computer 70, patient monitor 71 or cellular telephone 72, via the communication module 47 and link 48. The communication link 48 preferably is a wireless link, such as a radio frequency signal or a cellular telephone call, however it can be a cable that is occasionally plugged into the power source 14.

If the data indicates a serious abnormality in the patient, the signal from the power source 14 on communication link 48 alerts a caregiver to that condition. For this function the implant monitor 43 in the power source 14 shown in FIG. 3, analyzes the data that either was transferred from the medical device or which was derived from that transferred data. That analysis compares the data to setpoints previously stored in memory 44 which designate a condition or event that requires alerting medical personnel. The setpoints can be stored by the manufacturer of the tissue stimulation system 10 or programmed into the power source 14 by the medical personnel. Some setpoints are thresholds of the data, such as a specific heart rate or blood pressure, while other setpoints are dependent variables such as a rate of change of a type of data, e.g. a maximum allowable heart rate change. When the setpoint comparison indicates an alert condition, the implant monitor 43 sends an alert signal to the control circuit 45 indicating the nature of the associated condition.

Other alert conditions relate to the performance of the tissue stimulation system 10. For example, if the power feedback module 41 determines that the voltage on the implanted storage device 54 is below an acceptable level or that the second RF wireless signal has a signal strength below an given level or no longer is being received, as occurs when the arm band 22 is removed, the appropriate alert signal is sent to the control circuit 45 in the power source. The power feedback module 41 may calculate the power consumption of the medical device 15 and issue another alert signal when too much power is being consumed.

The control circuit 45 responds in several ways to these alert signals. A local alert is issued to the patient 11 from an annunciator such as an audible device 74 and a visible indicator 76 on the armband 22 on which the power source 14 is mounted. The audible annunciation is either a simple alarm tone or a voice message that is either pre-recorded or computer generated. Other types of annunciator displays can be provided for alphanumeric text and images related to the alert condition.

For example, an audible signal indicates when the power source 14 is at an optimal relative position with respect to the antenna assembly 24 of the implanted medical device 15. This function is initiated by closing a switch 78 on the power source 14. The RF communication receiver 42 in the power source 14 measures the strength of the second wireless signal 38 from the medical device 15 and a the audible device 74 emits a tone the loudness of which is varied in proportion to the strength of the second wireless signal. The best component positioning occurs when that signal strength is the greatest and is thus indicated when the tone is the loudest.

Remote alert annunciation also is provided to alert medical personnel such as a nurse, a caregiver, or a physician, or to alert a relative or another person. This further altering is carried out by the control circuit 45 forming a message based on the alert signal received from the implant monitor 43 or the power feedback module 41. That message is customized for the remote monitor that is to receive the alert. For the personal computer 70 or the patient monitor 71 the message can simply be a number indicating the specific condition that triggered the alert, e.g. non-receipt of the second RF signal or high blood pressure. Alternatively, the alert message provides more specific information such as the patient's blood pressure measurement that was too high. Upon receiving the message, the personal computer 70 or the patient monitor 71 decodes the message contents using a data table stored in that recipient device and uses other stored information to present text on its display screen to inform a person about the nature of the alert. For the cellular telephone 72, the control circuit formulates an audio message using pre-recorded announcements for the various alert conditions and sends that audio message to the communication module 47, which in this case is a cellular telephone. The communication module 47 dials a predefined telephone number and when the recipient telephone 72 is answered the audio message is sent over the telephone link.

The alerting is a multi-tier system for certain conditions which trigger an alert. For example, as noted previously the power source 14 issues an alert when the radio frequency second wireless signal 38 is not received from the implanted medical device 15, as occurs when the patient removes the arm band 22. This event initially causes the power source 14 to issue local alerts by activating the audible device 74 and the visible indicator 76. If within a given time period those alerts do not result in corrective action that reestablishes receiving the second wireless signal 38 (e.g. the patient putting on the arm band), the power source 14 issues an alert message via the communication module 47 to the remote monitors 70-72.

The loss of the second wireless signal 38 is considered a serious condition of the patient as it may result from deactivation of the tissue stimulation system 10. Examples of other serious conditions are excessively high blood pressure, absence of heartbeat for a prolonged time, and atrial fibrillation. In these cases, alert messages are issued immediately to the remote devices, without waiting to see if a local alert results in corrective action.

The present system provides impromptu situation-based, autonomous alerting by the tissue stimulation system 10 that allows corrective action at a tiered level, commensurate to the condition which triggered the alert. In autonomous alerting, the device takes action based on a set of criteria and circumstance. In some embodiments, environmental variables, such as air pressure, air temperature and skin temperature may be incorporated to correlate with physiological data prior to an alerting decision being made.

The alerting system is capable of self monitoring, physiological monitoring and autonomously alerting the patient, a bystander, a remote expert, a networked computer, a service person or a relative. Thus it is further intended to include alerting mechanism to communicate with different, independent communicable targets based on both the needs of the device and the patient based on predetermined conditions. In a first case, a caretaker can be alerted if internal and external components do not communicate with each other for a predetermined time. In a second case, the alerting mechanism may contact a medical service or physician if abnormal heart rhythms are observed. In a third example, the alerting mechanism may trigger a service call if communication is present but battery power is lower than a predetermined value.

The foregoing description was primarily directed to a preferred embodiment of the invention. Although some attention was given to various alternatives within the scope of the invention, it is anticipated that one skilled in the art will likely realize additional alternatives that are now apparent from disclosure of embodiments of the invention. Accordingly, the scope of the invention should be determined from the following claims and not limited by the above disclosure.

1. A medical apparatus for therapeutically treating a patient, said medical apparatus comprising:
a medical device for implantation entirely in vasculature of the patient and having a discriminator that receives and extracts energy from a first wireless signal which energy is used to power the medical device, a detector circuit that senses at least one of a physiological characteristic and performance of the medical device and in response produces data, and a feedback transmitter that sends information related to the data via a second wireless signal; and
an external power source, that is outside the patient and which transmits the first wireless signal, the external power source having a receiver that receives the second wireless signal and extracts the information, and a communication module with cellular telephone circuitry; wherein when the information indicates existence of a predefined condition, the communication module dials a predefined telephone number assigned to a remote monitor and sends an alert message for reception by the remote monitor.

2. The system as recited in claim 1 further comprising a remote monitor, at a location remote from the medical patient, and which receives the alert message and in response thereto alerts medical personnel.

3. The medical apparatus as recited in claim 1 wherein the medical device further comprises a pair of electrodes for contacting tissue of the patient, and a stimulation circuit that applies electrical stimulation pulses to the pair of electrodes.

4. The medical apparatus as recited in claim 3 wherein one of the medical device and the external power source analyzes the data to determine efficacy of tissue stimulation.

5. The medical apparatus as recited in claim 3 wherein the detector circuit is connected to the pair of electrodes and senses a physiological characteristic of the medical patient simultaneously when the electrical stimulation pulses are being applied to the pair of electrodes and produces the data from such sensing.

6. The medical apparatus as recited in claim 5 wherein the detector circuit is connected to the pair of electrodes and produces data that indicates effects of the electrical stimulation pulses on the patient.

7. The medical apparatus as recited in claim 3 wherein the detector circuit has an instrumentation amplifier with a variable gain and inputs connected to the pair of electrodes.

8. The medical apparatus as recited in claim 7 wherein the instrumentation amplifier has a lower gain while a stimulation pulse is being applied to the pair of electrodes than at other times.

9. The medical apparatus as recited in claim 1 wherein the data denotes a trend of the physiological characteristic.

10. The medical apparatus as recited in claim 1 wherein the medical device compares the data to a reference to determine existence of the predefined condition.

11. The medical apparatus as recited in claim 1 wherein the external power source compares the information to a reference to determine existence of the predefined condition.

12. The medical apparatus as recited in claim 1 wherein the external power source issues an alert indication when the second wireless signal has a signal strength that is below a given level.

13. The medical apparatus as recited in claim 1 wherein the external power source comprises an annunciator that indicates occurrence of the predefined condition.

14. The medical apparatus as recited in claim 1 wherein the external power source comprises an annunciator that indicates when the external power source is optimally positioned for communication with the medical device.

15. A medical apparatus that monitors a medical patient and stimulates tissue of the medical patient, said medical device comprising:
a medical device for implantation entirely in vasculature of the patient and having a discriminator that receives and extracts energy from a first wireless signal which energy is used to power the medical device, a pair of electrodes for contacting the tissue, a stimulation circuit that applies electrical stimulation pulses to the pair of electrodes, a detector circuit connected to the pair of electrodes to sense a physiological characteristic of the medical patient simultaneously when one of the electrical stimulation pulses is being applied to the pair of electrodes and produce data from such sensing, and a feedback transmitter that sends the data via a second wireless signal; and
an external power source that is outside the patient and which transmits the first wireless signal, the external power source having a receiver that extracts the data from the second wireless signal, and having an implant monitor which processes the data and provides an indication when the data indicates existence of a predefined condition, and a communication module that responds to the indication by sending a third wireless signal.

16. The medical apparatus as recited in claim 15 wherein the detector circuit produces data that indicates effects of the electrical stimulation pulse on the patient.

17. The medical apparatus as recited in claim 15 wherein the detector circuit has an instrumentation amplifier with a variable gain and inputs connected to the pair of electrodes.

18. The medical apparatus as recited in claim 17 wherein the instrumentation amplifier has a lower gain while a stimulation pulse is being applied to the pair of electrodes than at other times.

19. The medical apparatus as recited in claim 15 further comprising a remote monitor at a location remote from the medical patient which receives the third wireless signal and in response thereto issues an alert to medical personnel.

20. The medical apparatus as recited in claim 15 wherein the external power source issues an alert indication when the second wireless signal has a signal strength that is below a given level.

21. The medical apparatus as recited in claim 15 wherein the communication module has cellular telephone circuitry that produces the third wireless signal, wherein when the data indicates existence of a predefined condition, the communication module dials a telephone number assigned to a remote monitor and sends an alert message for reception by the remote monitor.

22. A medical apparatus for therapeutically treating a patient, said medical apparatus comprising:
a medical device for implantation entirely in vasculature of the patient and having a discriminator that receives and extracts energy from a first wireless signal which energy is used to power the medical device, a detector circuit that senses at least one of a physiological characteristic and performance of the medical device and in response produces data, and a feedback transmitter that sends information related to the data via a second wireless signal; and
an external power source, that is outside the patient and which transmits the first wireless signal, the external power source having a receiver that receives the second wireless signal, and an annunciator that in response to the second wireless signal indicates when the external power source is optimally positioned for communication with the medical device.

23. The medical apparatus as recited in claim 22 wherein the receiver in the external power source extracts the information from the second wireless signal.
